(19) United States
(12) Patent Application Publication
(10) Pub. No.: US 2014/0273254 A1
(43) Pub. Date: Sep. 18, 2014

(54) METHOD, PRODUCT, AND SYSTEM FOR QUANTIFYING THE METHYLATION STATUS OF A NUCLEIC ACID IN A SAMPLE

(71) Applicant: Thermo Fisher Scientific Oy, Vantaa (FI)

(72) Inventor: Jaakko Kurkela, Espoo (FI)

(73) Assignee: Thermo Fisher Scientific Oy, Vantaa (FI)

(21) Appl. No.: 14/212,416

(22) Filed: Mar. 14, 2014

Related U.S. Application Data
(60) Provisional application No. 61/788,239, filed on Mar. 15, 2013.

(51) Int. Cl. C12Q 1/68

(52) U.S. Cl. CPC C12Q 1/6827
USPC 436/94; 702/19

(57) ABSTRACT

Methods and systems are described for quantifying the methylation status of nucleic acids in a sample utilizing standardized curves derived from methylation-sensitive HRM data. The standardized curves are generated from HRM curves from a plurality of samples, each sample having a known but different methylation status. In one embodiment, the first negative derivative of each HRM curve from the known samples is plotted and a first value corresponding with a first melt peak and a second value corresponding with a second melt peak from the negative derivative plots are identified. The slope of a line connecting the first and second values for each sample is calculated and used to identify a slope data point that is plotted to generate the standardized curve. In another embodiment, a threshold line that intersects the plurality of HRM curves is generated and the standardized curve is generated from the intersection data points.

### CROSS-REFERENCE TO RELATED APPLICATIONS

This application claims the benefit of and priority to prior filed pending Provisional Application Ser. No. 61/788,239, filed Mar. 15, 2013, which is expressly incorporated herein by reference in its entirety.

### FIELD

The present invention relates generally to the analysis of double-stranded nucleic acids and, more particularly, to the quantification of the methylation status of nucleic acids in a sample by high resolution melt analysis of double-stranded nucleic acids.

### BACKGROUND

DNA methylation of cytosine residues is an epigenetic mechanism important in regulating gene expression and chromatin structure. DNA methylation plays a critical role in many normal and disease related processes and as such, quantification of DNA methylation is important in understanding these processes. Previous methods for quantifying DNA methylation typically involved a three-step procedure of DNA modification, PCR amplification, and analysis of PCR product. DNA modification involved treatment with a methylation-sensitive restriction endonuclease (“MSRE”) or sodium bisulfite prior to amplification. MSRE treatment cleaves non-methylated DNA while leaving methylated DNA intact. As such, only methylated DNA will be amplified in the subsequent PCR amplification step. Bisulfite treatment deaminates unmethylated cytosine to form uracil, while methylated cytosine remains unaffected. Bisulfite treated DNA can be analyzed by sequencing, methylation-specific PCR, and methylation-sensitive single nucleotide primer extension.

A more recently described method of analyzing the methylation status of nucleic acids utilizes methylation-sensitive high resolution melt analysis (HRM). Typically, for methylation-sensitive HRM analysis, a sample containing nucleic acids is treated with bisulfite. The bisulfate treated nucleic acid sequence is amplified using the PCR technique in the presence of a reporter molecule, such as a fluorescent dye, that selectively fluoresces when associated with a double-stranded nucleic acid. During PCR amplification, uracil from the bisulfate treatment is converted to thymine. The amplified sequence is subjected to HRM analysis. HRM analysis produces a generally sigmoid-shaped curve in which the signal level from the reporter molecule decreases as a function of temperature. The shape of the HRM curve and the melt temperature of the sample is determined by the specific sequence of nucleotides composing double-stranded nucleic acid. Samples treated with bisulfate will exhibit changes in the shape of the HRM curve and/or a shift in the melting temperature that correlates to the methylation status of the sample due to the change in the nucleic acid sequence caused by the differential effect of bisulfate on methylated and unmethylated cytosine residues.

While methylation-sensitive HRM is useful in identifying the presence or absence of methylation in a sample, quantification of the methylation status of nucleic acids in the sample has generally been subjectively determined by researcher. Typically, the researcher will visually compare the methylation-sensitive HRM curve from a test sample with methylation-sensitive HRM curves from samples with known methylation statuses to estimate the methylation status, such as the percent methylation in the test sample. While this method can allow for a general characterization of the methylation status in the test sample, its results are subjective, not easily reconcilable between experiments, and not capable of being automated. Moreover, subtle shifts in the melting temperature or shape of the methylation-sensitive HRM curve are not always easily determined or quantified. A need for a better method of analyzing methylation-sensitive HRM data was thus identified.

### SUMMARY

Methods are needed to accurately quantify the methylation status of nucleic acids in a sample. To this end, described herein are methods, systems, and program products for quantifying the methylation status of a nucleic acid in a sample utilizing standardized curves derived from methylation-sensitive HRM data.

In an embodiment, the method includes obtaining a plurality of curves from HRM data from a plurality of samples, where each sample has a known methylation status that is different from the methylation status of the other known samples. The method further includes plotting the first negative derivative of the HRM curves from the known samples and identifying a first value corresponding with a first melt peak and a second value corresponding with a second melt peak from the first negative derivative plot for each sample. The slope of a line connecting the first and second values for each sample is calculated. A slope data point for each sample is identified and plotted to generate the standardized curve. The standardized curve may be used to calculate the methylation status of a sample having an unknown methylation status. An embodiment of the method includes obtaining an HRM curve for a sample with the unknown methylation status and analyzing the HRM curve as described above to identify a slope data point for the sample and comparing the slope data point from the unknown sample with the standardized curve to quantify the methylation status of the unknown sample.

In another embodiment, the methylation status of a nucleic sample is quantified by a method that includes generating a standardized curve from a series of HRM curves with a threshold line that intersects the HRM curves. The method includes obtaining a plurality of curves from HRM data from a plurality of samples, where each sample has a known methylation status that is different from the methylation status of the other known samples. A threshold line that intersects the plurality of HRM curves is generated and an intersection data point is identified for each sample. The intersection data point for each of the plurality of HRM curves is plotted to generate the standardized curve. The standardized curve may be used to calculate the methylation status of a sample having an unknown methylation status. An embodiment of the method includes obtaining an HRM curve for a sample with an unknown methylation status and analyzing the HRM curve as described above to identify an intersection data point for the unknown sample. The intersection data point from the unknown sample is compared with the standardized curve to quantify the methylation status of the unknown sample.

### BRIEF DESCRIPTION OF THE DRAWINGS

The accompanying drawings, which are incorporated in and constitute a part of this specification, illustrate various embodiments of the invention and, together with a general description of the invention given above and the detailed description of the embodiments given below, serve to explain the embodiments of the invention.

FIG. 1A is a graph illustrating exemplary curves generated from methylation-sensitive HRM data in accordance with embodiments of the invention.

FIG. 1B is a graph illustrating exemplary plots of the first negative derivative of the HRM curves of FIG. 1A in accordance with embodiments of the invention.

FIG. 1C is a scatter plot of data points derived from a first negative derivative plot in accordance with embodiments of the invention.

FIG. 1D is a standardized curve derived from analysis of methylation-sensitive HRM data in accordance with embodiments of the invention.

FIG. 2 is a flow chart illustrating a process for generating a standardized curve from HRM data in accordance with embodiments of the invention.

FIG. 3 is a flow chart illustrating a process for generating an HRM curve in accordance with embodiments of the invention.

FIG. 4 is a flow chart illustrating a process for identifying a first peak and a second peak from the first negative derivative of an HRM curve in accordance with embodiments of the invention.

FIG. 5 is a flow chart illustrating a process for determining the methylation status of a nucleic acid in a sample in accordance with embodiments of the invention.

FIG. 6A is a graph illustrating exemplary methylation-sensitive HRM curves from data in accordance with embodiments of the invention.

FIG. 6B is a standardized curve derived from analysis of methylation-sensitive HRM data in accordance with embodiments of the invention.

FIG. 7 is a flow chart illustrating a process for generating a standardized curve from HRM data in accordance with embodiments of the invention.

FIG. 8 is a flow chart illustrating a process for determining the methylation status of a nucleic acid in a sample in accordance with embodiments of the invention.

FIG. 9 is a block diagram of a computer system in accordance with embodiments of the invention.

### DETAILED DESCRIPTION

Differences in the methylation status, such as the ratio of methylated to unmethylated nucleic acids, between samples can result in a shift, change in shape, or both a shift and a change in the shape of methylation-sensitive high resolution melt (“HRM”) curves generated with HRM data collected from such samples. Methylation-sensitive HRM may be used to analyze changes in the melt temperature of a nucleic acid sequence to infer the methylation status of the sequence. Typically, the sample nucleic acid is treated with the bisulfite to convert unmethylated cytosine to uracil, while not affecting methylated cytosine. Bisulfite treatment results in a nucleic acid sequence that is different from the sequence of the original sample, but that is determined by the methylation status of the nucleic acids in the original sample. The bisulfite treated target nucleic acid is then amplified using polymerase chain reaction (PCR). HRM data are then collected with the amplified bisulfite treated sequence.

HRM analysis is based on the principal that complimentary strands of nucleic acids form relatively stable double strands of nucleic acids at lower temperatures. As the temperature of a sample containing a double-stranded nucleic acid is increased, the double-stranded nucleic acid melts into two single strands. Similarly, as the temperature of a sample containing complimentary single strands of nucleic acid is decreased, the complimentary nucleic acids will reassociate into double-stranded nucleic acids. The melt temperature of a double-stranded nucleic acid, i.e., the temperature at which a nucleic acid transitions between a double-stranded nucleic acid and a pair of single strands, is determined by the length and sequence of the nucleic acid strands. Differences between two or more double-stranded nucleic acids, such as double-stranded nucleic acids amplified using PCR, may be inferred by observing and analyzing the high resolution melting or high resolution reassociation of the double-stranded nucleic acids over a range of temperatures. As used herein, the terms “high resolution melting” or “high resolution melt” are understood to include both high resolution melt and high resolution reassociation.

With reference to FIG. 1A, and in accordance with embodiments of the invention, an improved method of analyzing methylation-sensitive HRM data includes analyzing data collected from the high resolution melting of an amplified bisulfite treated nucleic acid sample wherein the data is characterized by a descending HRM curve. Similarly, the HRM data may be collected from the high resolution reassociation of a sample that includes complimentary single strands of a double-stranded nucleic acid. High resolution reassociation HRM data are characterized by an ascending HRM curve (not shown). The HRM curve includes a temperature value along the x-axis and a signal value representative of the concentration of double-stranded nucleic acid in the sample at a given temperature value along the y-axis. In an exemplary embodiment, the temperature value is expressed in degrees Celsius and the signal value is expressed as relative fluorescent units (RFU's).

In an exemplary embodiment, the signal value for a sample is obtained with a reporter molecule that selectively fluoresces when associated with a doubled stranded nucleic acid. Thus, the signal value, i.e., the level of fluorescence observed in a sample, is indicative of the concentration of double-stranded nucleic acid in the sample. Reporter molecules useful with embodiments of the invention described herein are those that selectively provide a signal, such as a fluorescent signal, when associated with a double-stranded nucleic acid. For example, fluorescent double-stranded nucleic acid dyes used in real time PCR reactions may be used. Exemplary reporter molecules include SYBR® Green I, SYBR® Gold, PicoGreen® (each available from Invitrogen), and LC Green®, Eva Green, Melt Doctor, SYTO®-9, SYTO®-13, SYTO®-16, SYTO®-60, SYTO®-62, SYTO®-64, SYTO®-82, POPO-3, TOTO-3, PO-PRO-3, TO-PRO-3, YO-PRO®-1, SYTOX® Orange, BEBO, BOXTO, Chromofy, as well as other reporter molecules that selectively fluoresce when associated with double-stranded nucleic acids. In addition to the use of reporter molecules that selectively associate with double stranded nucleic acids, the reporter molecules may also be associated with fluorescent probes or primer based systems. As used herein, the term reporter molecule is understood to include any system, molecule, probe, dye, or combination thereof that is capable of generating a signal that corresponds to the concentration of double-stranded nucleic acid in a sample at a particular temperature.

For high resolution melting, the signal value is obtained from measurements taken at predetermined increments as the temperature of the sample is slowly increased from a temperature at which substantially all of the complementary nucleic acid strands in the sample are in the double-stranded state, to a temperature at which no double-stranded nucleic acid is detectable with the reporter molecule. For high resolution reassociation, the signal value is obtained from measurements taken at predetermined increments as the temperature of the sample is slowly decreased from a temperature at which substantially all of the complementary nucleic acid strands in the sample are in the single-stranded state, to a temperature at which substantially all of the nucleic acid is in a double-stranded state as detected with the reporter molecule. Typically, the signal value is measured over a range of temperatures from about 60 degrees Celsius to about 95 degrees Celsius; however, the temperature range may be increased or decreased as needed to analyze a specific nucleic acid sequence.

In accordance with embodiments of the invention, the signal value is obtained as the temperature increases by fractions of a degree over at least a portion of the melting temperature range. In an embodiment, the signal value is obtained at about every 0.1 degrees Celsius over at least a portion of the melting temperature range. In an alternative embodiment, the signal value is obtained at about every 0.2 degrees Celsius over at least a portion of the melting temperature range. In an alternative embodiment, the signal value may be obtained at about every 0.04 degrees Celsius to about 5.0 degrees Celsius over at least a portion of the melting temperature range.

With reference to FIGS. 1A and 6A, the data obtained from HRM analysis of a double-stranded nucleic acid generally forms a sigmoid shaped curve having a saturation region, a melt region, and a background region. The saturation region is a relatively flat region typically at lower temperature values in the curve and characterized by high signal values because the double-stranded nucleic acid concentration in the sample is constant because at these temperatures the concentration of double-stranded nucleic acid in the sample does not change. Thus, in the saturation region, there are no changes in the signal generated by the reporter molecule that are related to changes in the concentration of double-stranded nucleic acids. In some embodiments, the double-stranded nucleic acid is saturated with reporter molecules whereas in other embodiments, the double-stranded nucleic acid is not saturated. The region of exponential melting is the central region of the curve wherein the signal value generated by the reporter molecule changes in relation to exponential changes in the concentration of double-stranded nucleic acid as the double-stranded nucleic acid melts or reassociates with changes in the temperature. The background region is a relatively flat region typically at higher temperature values. In the background region, the concentration of double-stranded nucleic acids in the sample is reduced to levels that the signal from the reporter molecule associated with double-stranded nucleic acids cannot overcome the background signals in the reaction chamber.

In accordance with embodiments of the invention, HRM curves, such as shown in FIGS. 1A and 6A, are generally obtained from a plurality of samples wherein each samples has a different known methylation status, such as a different known ratio of methylated to unmethylated nucleic acids. For example, the plurality of samples with known methylation status can include a series of samples having increasing or decreasing levels of methylation, such as no methylation, 0.1 percent methylation, 1 percent methylation, 10 percent methylation, 50 percent methylation, and 100 percent methylation. The HRM curves are used to generate a standardized curve based on the differences between the HRM curves attributed to the differences in nucleic acid sequence that correspond to the methylation status of the sequence, such as the differences introduced to the nucleic acid sequence by bisulfate treatment. Differences between HRM curves generated from the samples may be observed as differences in the melt temperature of the double-stranded nucleic acids from the samples, differences in the shape of the HRM curves, or differences in both the melt temperatures and the shapes of the HRM curves. The melt temperature is the temperature at which the greatest amount of double-stranded nucleic acid melts. For HRM curve analysis, the melt temperature is the temperature at which the absolute value of the slope in the region of exponential melting is the greatest. A sample having a mixture of two or more amplified sequences resulting from differences in the methylation status of the original sample, may have at least two melt temperatures, one melt temperature for each amplified sequence.

In addition, when a double stranded nucleic acid is subjected to gradual heating, discrete domains in the double stranded nucleic acid sequence may melt in steps based on the sequence of nucleic acids in the domain. Thus, some domains of a double stranded nucleic acid may have a lower melting temperature when compared to other domains of the same nucleic acid sequence. The different melting temperatures for the two or more domains may affect the shape of the resulting HRM curve. The different melting temperatures for the two or more domains in a nucleic acid sequence may be identified by analyzing the HRM curve to identify two or more regions of exponential melting. These differences may be used to analyze the methylation status of a sample.

With reference to FIGS. 1A and 6A, exemplary HRM curves demonstrate a rightward shift and differences in the shape of HRM curves as the percent of methylated residues in the samples increases. One of ordinary skill will appreciate that changes in the methylation status of a nucleic acid sequence could result in a leftward shift in the HRM curves (not shown). These differences can be exploited to generate standardized curves from a plurality of samples having known but different methylation statuses, such as known ratios of methylated to unmethylated nucleic acids, to allow for the quantification of the methylation status in a sample in which the methylation status of the nucleic acid is not known. Thus, embodiments of the invention are directed to methods of quantifying the methylation status in a sample by generating a standardized curve based on the differences in HRM curves generated with a plurality of samples, each sample having a known but different methylation status, such as different ratios of methylated to unmethylated nucleic acids.

With reference to FIG. 2, an aspect of the invention is directed to a method that generally includes obtaining an HRM curve with a sample having a known methylation status (block 110), plotting the first negative derivative of the HRM curve (block 112), detecting a first value associated with a first melt peak and a second value associated with a second melt peak of the first negative derivative plot (block 114), calculating the slope of a line connecting the first and second values (block 116), identifying a slope data point based on the calculated slope (block 120), and plotting the slope data point on a standardized curve (block 124). This general process is repeated for additional samples having a known methylation status (block 126) such that the standardized curve includes a series of slope data points calculated from samples with each sample having a different known methylation status, such as different ratio of methylated to unmethylated nucleic acids. This general process is also repeated with a sample having an unknown methylation status where the slope calculated for the sample with the unknown methylation status is compared to the standardized curve to identify the methylation status of nucleic acids in the unknown sample, as discussed in greater detail below with reference to FIG. 5.

FIG. 3 illustrates an exemplary method of generating an HRM curve for analysis in accordance with embodiments of the invention. First, an HRM curve is obtained from HRM data gathered from the high resolution melting or high resolution reassociation of a sample containing a double-stranded nucleic acid and a reporter molecule (block 128). The HRM data includes a series of data points having a signal value and a temperature value. The HRM data may be obtained from an HRM analysis system, such as one that includes a thermal cycler for heating and/or cooling the sample in a controlled manner and an optical system for obtaining signal values as the sample is heated or cooled. The HRM data may optionally be internally smoothed (block 130), have the exponential decay removed (block 132), be normalized relative to other HRM curves (block 134) or combinations of internal smoothing, removing the exponential decay, and normalization.

The optional internal smoothing process (block 130) may employ any process that internally removes insignificant variations in the data that are not associated with changes in the concentration of double-stranded nucleic acids. For example, in one embodiment, the smoothing process employs a rolling average method that averages the product values for a plurality of consecutive data points from the HRM data. In another embodiment, the data are smoothed with a Savitzky-Golay smoothing filter by fitting an n^{th} degree polynome to a plurality of consecutive data points and calculating a smoothed product value for one or several data points with the plurality of data points. In one embodiment, the user may optionally designate the number of data points used for the rolling average.

The optional exponential decay removal (block 132) process removes decreasing signal value trends that are not related to changes in the double-stranded nucleic acid concentration. Exponential decay can be removed by known processes, such as mathematical processes that calculate the amount of decay observed in the saturation region of the HRM curve. For example, a line segment may be fit by linear regression to a subset of data points in the saturation region. The slope of the line segment may then be used to correct the HRM curve. In another example, the exponential decay is removed from the curve directly by multiplying the measured melting curve by a correction function which is exponentially dependent on the temperature.

The optional normalizing step removes variability in the first and second peaks that is not associated with the double-stranded nucleic acids in the samples. For example, the first and second melt peaks can vary due to the position of the reaction well on the thermal block or due to inaccuracies in measuring the reagents used in the analysis. In an embodiment, the HRM curves from a plurality of samples are normalized relative to one another before plotting the first negative derivative for each HRM curve to account for at least a portion of the variability in the melt peaks that is not associated with the double-stranded nucleic acids in the samples (FIG. 3, block 134). Normalizing the HRM curves results in first negative derivative plots that have identical areas under the curves. Because the area under the curve for the normalized plots are identical, any significant difference in the melt peaks for each plot indicates a difference in the shapes of the underlying HRM curves and, correspondingly, indicates a difference in the double-stranded nucleic acids from which the HRM curves originate. For this embodiment, HRM curves are generated from the HRM data for each sample.

HRM data may be normalized by any process that normalizes the data along the thermal axis (x-axis), the signal axis (y-axis) or along both the thermal axis and the signal axis. For thermal axis normalization, each HRM curve is shifted on the thermal axis based its location on the thermal block as determined by the thermal characteristics of the thermal block. For example, the detected melt temperature for each well may be multiplied by a standard adjustment multiplier that corresponds to the typical variation of that well from the mean of the block. The signal axis may be normalized based on user defined areas of interest in the saturation region and the background regions or preliminary areas of interest in these regions may be automatically calculated. In one embodiment, the areas of interest are identified from a first negative derivative plot of the HRM curve. The areas of interest are the areas of the first negative derivative plot having low values that correspond to areas of the HRM curves wherein the change in slope is small. The same area of interest is used for all curves being normalized to one another. The average signal value in the areas of interest across all curves being normalized are averaged and set to a first normalized signal value, such as 100, for the area of interest associated with the saturation region, and a second normalized value, such as 0, for the area of interest associated with the background region. The remaining data points are normalized to relative to the first normalized signal value and the second normalized signal value.

After obtaining the HRM curve for a sample having a known methylation status, such as a known ratio or methylated to unmethylated nucleic acids, and optionally smoothing the data, removing the exponential decay and normalizing the curves relative to one another, the first negative derivative is plotted for each HRM curve (block 112 of FIG. 2) and the first negative derivative plot is analyzed to detect its first and second melt peaks (block 114).

FIG. 1B illustrates exemplary first negative derivative plots from the HRM curves from FIG. 1A. The first negative derivative plot for each sample generally has eight regions: (1) a first background region, (2) a first ascending region, (3) a first melt peak, (4) a first descending region, (5) a second ascending region, (6) a second melt peak, (7) a second descending region, and (8) a second background region. The first negative derivative plot may include additional melt peaks as well wherein each peak would include an ascending region, a melt peak, and a descending region. In some instances, the first and second melt peaks may mask one another such that the first negative derivative plot of a sample appears to have a single melt peak. When this occurs, the melt peaks may be unmasked, such as described herein. The first and second background regions of the first negative derivative plots illustrated in FIG. 1B correspond to the saturation region and background region, respectively, of the corresponding HRM curves of FIG. 1A. The first and second ascending regions and the first and second descending regions of the first negative derivative plot of FIG. 1B correspond to portions of the exponential melting region of the HRM curves of FIG. 1A. The melt peaks of the first negative derivative plots of FIG. 1B corresponds to the points along the exponential melting region of the HRM curves of FIG. 1A having the steepest slope relative to surrounding data points in the exponential region.

As illustrated in FIG. 1B, in an embodiment, the first melt peak is identified from the first negative derivative plot as the data point having the greatest amplitude, i.e., the greatest distance from the x-axis where the x-axis value is zero. The melt peak has a height value that corresponds to the first negative derivative of the signal value and a temperature value that corresponds with the melt temperature for the sample.

In an alternative embodiment illustrated in FIG. 4, the first melt peak is identified from a Gaussian probability function that is fit to the first negative derivative curve (block 140). The first melt peak of the first Gaussian probability function is identified as the point along the function having the greatest amplitude, i.e., the greatest height or distance from the x-axis where the x-axis value is zero (block 142). The first Gaussian probability function is then subtracted from the first negative derivative plot (block 144). The second melt peak may then be identified as the data point along the subtracted data set having the greatest amplitude. In the alternative, the second melt peak may be identified from the subtracted data set by fitting a second Gaussian probability function to the subtracted curve (block 146). In this alternative, the second melt peak is identified as the data point along the second Gaussian probability function having the greatest amplitude (block 148).

The melt peaks generally have a temperature value along the thermal axis (i.e., the x-axis) corresponding to the temperature of the sample and a signal value along the signal axis (i.e., the y-axis) corresponding to the concentration of double stranded nucleic acids in the sample.

With reference to FIG. 2, the first melt peak is used to identify a first value that corresponds with the first melt peak and the second melt peak is used to identify a second value that corresponds with the second melt peak (block 114). The first value and the second value may be a peak height value, a width value, an area under the curve value, and combinations thereof that are associated with the first and second melt peaks.

The peak height value may be a point along one of the first negative derivative plot, the subtracted data, or a Gaussian probability function fit to the first negative plot or subtracted data having the greatest amplitude.

The width value may be identified from the first negative derivative plot, the subtracted data, or a Gaussian probability function fit to the first negative plot or subtracted data as the width of the curve at a specified fraction of the melt peak height. In one embodiment, the width value is determined at about fifty percent of the melt peak height. In an alternative embodiment, the width value is determined at an optimum fraction of the melt peak height that is selected from the range between about 15 percent and about 85 percent of the melt peak height. The same fraction of the melt peak height is used to calculate or measure the width value of the curves for each melt peak and across all samples.

The area under the curve (AUC) value may be identified from the first negative derivative plot, the subtracted data, or a Gaussian probability function fit to the first negative plot or subtracted data as the AUC at a specified fraction of the melt peak height. In one embodiment, the AUC value is determined at about fifty percent of the melt peak height. In an alternative embodiment, the AUC value is determined at an optimum fraction of the melt peak height that is selected from the range between about 15 percent and about 85 percent of the melt peak height. The same fraction of the melt peak height is used to calculate or measure the AUC value of the curves for each melt peak and across all samples.

In an embodiment, the first and second values that correspond to the first and second melt peaks, respectively, each generally include two values selected from the group consisting of a peak height value, a width value, an AUC value and a temperature value. With reference to FIG. 2, the method next calculates the slope of a line connecting the first value and the second value. The slope may be calculated by plotting the first value and second value on a scatter plot, connecting a line between the plotted first and second values, and measuring or calculating the slope of the line, as illustrated in FIG. 1C. In the alternative, the slope may be calculated directly from the first and second values without the need for separately plotting the first and second values on a scatter plot.

With reference again to FIG. 2, a slope data point is identified from the slope of a line connecting the first value and the second value (block 120). The slope data point generally includes two values with one value corresponding to the slope calculated in the previous step and the other value being associated with the methylation status of the nucleic acids in the sample.

The slope data point identified for each sample having a known methylation status, such as each known ratio of methylated to unmethylated nucleic acids, is plotted to generate a standardized curve (FIG. 2, block 124). In general, the slope data points from the samples are plotted and a curve is fit to the data points. FIG. 1D illustrates an exemplary standardized curve where the y-axis represents the slope of the line connecting the first value and the second value for each sample and the x-axis represents the methylation status such as a percent methylation representative of the ratio of methylated to unmethylated nucleic acids in the samples.

The method uses the standardized curve to calculate the methylation status in a sample where the methylation status is unknown. The unknown sample is analyzed in a manner similar to that describe above with respect to the analysis of samples having nucleic acids with a known methylation status. Specifically, with reference to FIG. 5, the analysis includes obtaining an HRM curve with a sample having an unknown methylation status (block 150), plotting the first negative derivative of the HRM curve (block 152), detecting a first value associated with a first melt peak and a second value associated with a second melt peak of the first negative derivative plot (block 154), calculating the slope of a line connecting the first and second values (block 156), and comparing the slope for the unknown sample with the standardized curve to identify the methylation status for the unknown sample (block 160). The first four steps may be practiced in the same manner as described above with respect to the samples having a known methylation status. For the comparing step, the slope from the unknown sample is compared to the standardized curve and a data point along the standardized curve having a slope value that matches the slope value from the unknown sample is identified. The methylation status for the unknown sample is determined from the methylation status value for the identified data point. Other methods for identifying the data point along the standardized curve that most closely matches the slope from the unknown sample could also be used such as calculating the methylation status based on the mathematical formula describing the standardized curve.

With reference to FIGS. 6A, 6B, and 7, another aspect of the invention is directed to a method of generating a standardized curve that includes obtaining a series of methylation-sensitive HRM curves wherein each HRM curve is generated from a sample having a different but known methylation status (FIG. 6A and blocks 170, 172, and 174 of FIG. 7), generating a threshold line that intersects each of the HRM curves at an intersection data point (FIG. 6A and block 176 of FIG. 7), and plotting the intersection data points to generate a standardized curve (FIG. 6B and block 178 of FIG. 7).

The methylation-sensitive HRM curves from methylation sensitive HRM data for each sample are generated as described above. With reference to FIG. 3, the HRM data may optionally be internally smoothed (block 130), have the exponential decay removed (block 132), be normalized relative to the other HRM curves (block 134) or combinations of internal smoothing, removing the exponential decay, and normalization.

In an exemplary embodiment, the standardized curve is generated from HRM curves from two or more samples, and preferably at least three samples wherein each sample has a different known methylation status. For example, a first sample may have a first methylation status, such as a first known ratio of methylated to unmethylated nucleic acids expressed as the percent methylation, the second sample will have a second methylation status that is greater than the methylation status of the first sample, and the third sample will have a third methylation status that is greater than the methylation status of the second sample. One of ordinary skill will appreciate that more samples could be used for the standardized curves and that the additional samples may each have a different methylation status from the other samples.

In an embodiment, the threshold line is generated by a processor system based on parameters that would result in an optimized standardized curve. For example, the processor system can generate a threshold line that results in a standardized curve, compare the generated standardized curve with a predetermined optimized standardized curve, and based on the comparison either use the original threshold line or generate a new optimized threshold line. The processor system could repeat this process until the threshold line generates an optimized standardized curve. For example, the processor may compare the mathematical formula describing the standardized curve with a mathematical formula describing a predetermined optimized standardized curve and based on this comparison, the processor may generate a new optimized standardize curve with a different angle, different origin, or both a different angle and different origin as compared to an axis of the plot. The processor can then compare the newly generated standardized curve with the predetermined optimized standardized curve to determine if a further round of optimization is needed.

As an alternative, the processor system can generate a threshold line that meets a predetermined criterion such as having a predetermine origin and a predetermined angle. As another alternative, the predetermined criterion may include the threshold line intersecting one or more of the HRM curves at a predetermined data point along the HRM curve. In another example the processor may generate a threshold line that intersects at least two of the HRM curves and preferably at least three of the HRM curves at the predetermined data point. The predetermined data point could be a percentage of the maximum value of the HRM curve, such as 50 percent of the maximum value or a range of data points having a value that ranges between 40 percent of the maximum and 60 percent of the maximum. The predetermined criterion may be user defined such as a user defined origin, angle, or data point along the HRM curves.

In an alternative embodiment, the threshold line is user generated. The user generated threshold allows the user to select a threshold line that results in a user optimized standardized curve such as one that matches details from a predetermined optimized standardized curve. The user defined threshold line could be defined by the origin, angle, or by using a computer interface, such as a mouse, to control the placement and angle of the threshold line.

The intersection data point for each HRM curve corresponds to the relative position of the HRM curve and the methylation status of the nucleic acid sample from which the HRM curve was generated. Accordingly, each intersection data point has a first value corresponding to the relative distance of the intersection data point from a reference data point and a second value corresponding to the methylation status of the nucleic acid sample from which the HRM curve was generated. The reference data point may be an intersection data point from the HRM curve generated from one of the known samples, such as the intersection data point from the sample having the lowest methylation status. In the alternative, the reference data point may be one of the origin of the threshold line, the x-intercept of the threshold line, the y-intercept of the threshold line, or a point along the threshold line wherein at least one of the first value or the second value is less than the respective first value or second value for the data point from the sample having the lowest methylation status.

With reference to FIG. 8, the standardized curve may be used to calculate the methylation status in a sample where the methylation status is unknown. Specifically, the method includes generating an HRM curve with a sample having an unknown methylation status (block 182), identifying the data point along the HRM curve for the unknown sample that is intersected by the threshold line utilized to generate the standardized curve (block 184), and comparing the intersection data point from the unknown sample with the standardized curve to identify the methylation status of the unknown sample (block 186). The first two steps may be practiced in the same manner as described above with respect to the samples having a known methylation status. For the comparing step, the relative position of the intersection data point from the unknown sample is compared to the standardized curve and a data point along the standardized curve having a relative position value that matches the relative position value from the unknown sample is identified. The methylation status for the unknown sample is determined from the methylation status value for the identified data point from the standardized curve. Other methods for identifying the data point along the standardized curve that most closely matches the relative position from the unknown sample could also be used such as calculating the methylation status based on the mathematical formula describing the standardized curve.

One of ordinary skill will appreciate that samples may be run in duplicate, triplicate, or more. The multiples of each sample may be considered individually or together such as by averaging before or after the generation of the HRM curves, or before or after the identification of a data point from the HRM curve that is used to generate the standardized curves.

With reference to FIG. 9, embodiments of the invention include a program code 200 that includes instructions executable on a processor system 202, such as a tablet, a computer, or computer system, for carrying out the steps of the method. In one embodiment, the program code 200 includes instructions for analyzing methylation-sensitive HRM data. Embodiments of the invention, whether implemented as part of an operating system 204, application, component, program code 200, object, module or sequence of instructions executed by one or more processing units 206 are referred to herein as “program code.” The program code 200 typically comprises one or more instructions that are resident at various times in various memory and storage devices 212 in the processor system 202 that, when read and executed by one or more processors 206 thereof cause that processor system 202 to perform the steps necessary to execute the instructions embodied in the program code 200 embodying the various aspects of the invention.

While embodiments of the invention are described in the context of fully functioning processing systems 202, those skilled in the art will appreciate that the various embodiments of the invention are capable of being distributed as a program product on a computer readable storage medium. The program product may embody a variety of forms. The invention applies equally regardless of the particular type of computer readable storage medium used to actually carry out the distribution of the program code 200. Examples of appropriate computer readable storage media for the program product include, but are not limited to, non-transitory recordable type media such as volatile and nonvolatile memory devices, floppy and other removable disks, hard disk drives, USB drives, optical disks (e.g. CD-ROM's, DVD's, Blu-Ray discs, etc.), among others.

Any of the individual processes described above or illustrated in the figures may be formed into routines, procedures, methods, modules, objects, and the like, as is well known in the art. It should be appreciated that embodiments of the invention are not limited to the specific organization and allocation of program functionality described herein. In addition, the systems for analyzing HRM data may further include a module for collecting the HRM data (i.e. a HRM data generator) 210, a module for receiving HRM data 216, and a display 214 for displaying information. The HRM data collection module may include a thermal cycler and a device for detecting the signal value that results from HRM analysis, such as a change in fluorescence from double-stranded nucleic acid over a range of temperatures. HRM data collection modules as known in the art may be used in accordance with the invention. The HRM data receiving module includes components and/or program code to receive HRM data from the HRM data collection module.

### Example 1

HRM curves were generated from a series of samples having a known percentage of methylated nucleic acids (FIG. 1A). The percent methylation corresponds to the total nucleic acids capable of being methylated in the sample sequence. HRM curves were generated for nucleic acid samples that were not methylated or that were 1 percent methylated, 10 percent methylated, 50 percent methylated, or 100 percent methylated. The samples were prepared by mixing an unmethylated nucleic acid sample with a bisulfite treated fully methylated nucleic acid sample at ratios that correspond to the desired percent methylation. HRM curves were generated for duplicate samples for each percentage of methylated nucleic acids. The HRM curve data was averaged for each duplicate and the first negative derivative was plotted for the averaged HRM curves (FIG. 1B). A Gaussian probability function was fit to each HRM curve and the peak of each Gaussian probability function was identified as the point along the Gaussian probability function having the greatest amplitude. The Gaussian probability function was subtracted from the first negative derivative plots for each sample and a second Gaussian probability function was fit to the subtracted data. A second melt peak was identified as the data point along the second Gaussian probability function having the greatest amplitude. For each sample, the two identified melt peak data points were plotted on a scatter plot and the slope of a line connecting the two data points was calculated (FIG. 1C). The slopes for each sample were used to identify a slope data point that included the slope value and the percent methylation for the sample. The slope data points were plotted and a standardized curve was fit to the data points (FIG. 1D).

### Example 2

HRM curves were generated from a series of samples having a known percentage of methylated nucleic acids (FIG. 1A). The percent methylation corresponds to the total nucleic acids capable of being methylated in the sample sequence. HRM curves were generated for nucleic acid samples that were not methylated, 1 percent methylated, 10 percent methylated, 50 percent methylated, or 100 percent methylated. The samples were prepared by mixing an unmethylated nucleic acid sample with a bisulfite treated fully methylated nucleic acid sample at ratios that correspond to the desired percent methylation. HRM curves were generated for duplicate samples for each percentage of methylated nucleic acids. A user generated threshold line that intersected each of the samples was plotted and the average of the intersections for each pair of duplicates was identified. The relative position of the intersection data point for each averaged known sample was identified relative to the distance of the respective data point from the averaged intersection data point for the unmethylated sample. The intersection data points were plotted and a standardized curve was fit to the plotted data points.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A method of analyzing HRM data to quantify the methylation status of nucleic acids in a sample comprising:
obtaining a first curve from HRM data from a first sample having a first known methylation status of nucleic acids,
plotting the first negative derivative of the first curve,
identifying a first value corresponding with a first melt peak and a second value corresponding with a second melt peak from the first curve,
calculating a first slope of a line connecting the first value with the second value from the first curve, and
identifying a first slope data point corresponding to the first slope and the first known methylation status of nucleic acids in the first sample;
obtaining a second curve from HRM data from a second sample having a second known methylation status of nucleic acids,
plotting the first negative derivative of the second curve,
identifying a first value corresponding with a first melt peak and a second value corresponding with a second melt peak from the second curve,
calculating a second slope of a line connecting the first value with the second value from the second curve, and
identifying a second slope data point corresponding to the second slope and the second known methylation status of nucleic acids in the second sample; and
generating a standard curve with the first slope data point and the second slope data point.

2. The method of claim 1 further comprising:
obtaining a third curve from HRM data from a third sample having an unknown methylation status of nucleic acids,
plotting the first negative derivative of the third curve,
identifying a first value corresponding with a first melt peak and a second value corresponding with a second melt peak from the third curve, and
calculating a third slope of a line connecting the first value with the second value from the third curve; and
comparing the third slope with the standard curve to quantify the methylation status of nucleic acids in the third sample.

3. The method of claim 2 wherein the methylation status of nucleic acids in the third sample is quantified by identifying the methylation status indicated by a data point along the standard curve that corresponds with the third slope.

4. The method of claim 1 further comprising at least one of smoothing the HRM data or removing exponential decay from the HRM data.

5. The method of claim 1 further comprising normalizing the curves for each sample relative to one another.

6. The method of claim 1 wherein the first value for a curve from a sample is selected from the group consisting of a peak height value, a width value, an area under the curve value, and combinations thereof identified from the first negative derivative plot.

7. The method of claim 6 wherein the peak height value is a data point along the subtracted data set having the greatest amplitude.

8. The method of claim 6 wherein the width value or the area under the curve value is calculated at about 50 percent of the peak height value.

9. The method of claim 6 wherein the width value or the area under the curve value is calculated at a fraction of the peak height value that is in the range between about 15 percent of the peak value and about 85 percent of the peak height value.

10. The method of claim 1 further comprising fitting a first Gaussian probably function to the first negative derivative plot for a curve from a sample; and
the first value is selected from the group consisting of a peak height value, a width value, an area under the curve value, and combinations thereof identified from the first Gaussian probability function.

11. The method of claim 10 further comprising subtracting the first Gaussian probability function from the first negative derivative plot and identifying the second value from the subtracted data set.

12. The method of claim 11 wherein the second value is selected from the group consisting of a peak height value, a width value, an area under the curve value, and combinations thereof identified from the first subtracted data set.

13. The method of claim 11 further comprising fitting a second Gaussian probability function to the subtracted data set; and
the second melt peak is selected from the group consisting of a peak height value, a width value, an area under the curve value, and combinations thereof identified from the second Gaussian probability function.

14. The method of claim 1 further comprising:
obtaining a fourth curve from HRM data from a fourth sample having a fourth known methylation status of nucleic acids,
plotting the first negative derivative of the fourth curve,
identifying a first value corresponding with a first melt peak and a second value corresponding with a second melt peak from the fourth curve,
calculating a fourth slope of a line connecting the first value with the second value from the fourth curve, and
identifying a fourth slope data point corresponding to the fourth slope and the fourth known methylation status of nucleic acids in the fourth sample; and
generating a standard curve with the first slope data point, the second slope data point, and the third slope data point.

15. The method of claim 14 further comprising:
obtaining a fifth curve from HRM data from a fifth sample having a fifth known methylation status of nucleic acids,
plotting the first negative derivative of the fifth curve,
identifying a first value corresponding with a first melt peak and a second value corresponding with a second melt peak from the fifth curve,
calculating a fourth slope of a line connecting the first value with the second value from the fifth curve, and
identifying a fifth slope data point corresponding to the fifth slope and the fifth known methylation status of nucleic acids in the fifth sample; and
generating a standard curve with the first slope data point, the second slope data point, the fourth slope data point, and fifth data point.

16. A system for practicing the method of claim 1, the system comprising a processor system and a program code for practicing each step of claim 1.

17. A program code product for practicing the methods of claim 1, the program code product comprising:
a computer readable storage medium; and
program instructions for performing the method of claim 1,
wherein the program instructions are stored on the computer readable storage medium.

18. A method of analyzing HRM data to quantify the methylation status of nucleic acids in a sample, the method comprising:
obtaining a first curve with HRM data collected from a first sample having a first known methylation status of nucleic acids;
obtaining a second curve with HRM data collected from a second sample having a second known methylation status of nucleic acids that is greater than the first known methylation status of nucleic acids;
obtaining a third curve with HRM data collected from a third sample having a third known methylation status of nucleic acids that is greater than the second known methylation status of nucleic acids;
generating a threshold line that intersects the first curve at a first intersection data point, the second curve at a second intersection data point, and the third curve at a third intersection data point; and
plotting a standard curve with the first data point, the second data point, and the third data point.

19. The method of claim 18 further comprising:
obtaining a fourth curve with HRM data collected from a fourth sample having an unknown methylation status of nucleic acids;
identifying a fourth data point that corresponds with the intersection of the threshold line with the fourth curve; and
comparing the fourth data point with the standard curve to quantify the methylation status of nucleic acids in the fourth sample.

20. The method of claim 18 wherein each of the first data point, the second data point, the third data point, and fourth data points have a first value and a second value, the first value corresponding to the relative distance of the respective data point from a reference data point and the second value corresponding to the methylation status of nucleic acids in the sample.

21. The method of claim 20 wherein the reference data point is selected from the group consisting of the first data point, the origin of the threshold line, the x-intercept of the threshold line, the y-intercept of the threshold line, and a point along the threshold line wherein at least one of the first value or the second value is less than the respective first value and second value for the first data point.

22. The method of claim 18 wherein the threshold line is generated by the user.

23. The method of claim 18 wherein the threshold line is generated by a processor.

24. A system for practicing the method of claim 18, the system comprising a processor system and a program code for practicing each step of claim 18.

25. A program code product for practicing the methods of claim 18, the program code product comprising:
a computer readable storage medium; and
program instructions for performing the method of claim 18,
wherein the program instructions are stored on the computer readable storage medium.
